(19) United States
(12) Patent Application Publication
(10) Pub. No.: US 2026/0283538 A1
(43) Pub. Date: Sep. 24, 2026

(54) Noninvasive Transabdominal Fetal Electroencephalography

(71) Applicant: Yale University, New Haven, CT (US)

(72) Inventors: Jose Cortes-Briones, New Haven, CT (US); Emily Lee, New Haven, CT (US)

(73) Assignee: Yale University, New Haven, CT (US)

(21) Appl. No.: 19/479,991

(22) PCT Filed: May 2, 2024
(86) PCT No.: PCT/US2024/027490

§ 371 Date: Oct. 29, 2025

Related U.S. Application Data
(60) Provisional application No. 63/464,341, filed on May 5, 2023.

(51) Int. Cl. A61B 5/00; A61B 5/369; G16H 50/20

(52) U.S. Cl. CPC A61B 5/4362; A61B 5/369; A61B 5/6823; A61B 5/7214; A61B 5/7267; G16H 50/20; A61B 2503/02

(57) ABSTRACT

Examples described herein provide a computer-implemented method that includes receiving a noninvasive transabdominal fetal electroencephalography (TA-fEEG) signal associated with a pregnant subject. The method further includes reducing unwanted noise in the TA-fEEG signal using a first machine learning model. The method further includes reconstructing a fetal electroencephalography (fEEG) signal from the TA-fEEG signal using a second machine learning model.

### CROSS-REFERENCE TO RELATED APPLICATION

The present application claims priority benefit to a U.S. provisional patent application entitled “Non-Invasive Transabdominal Fetal Electroencephalography,” which was filed on May 5, 2023, and assigned Ser. No. 63/464,341 The entire content of the foregoing U.S. provisional patent application is incorporated herein by reference.

### BACKGROUND

Electroencephalography (EEG) involves measuring electrical signals in a subject by placing electrodes on the subject. The electrodes measure signals, which can be presented as an electrogram useful for monitoring and diagnostic purposes. One use of EEGs is to measure brain activity of a subject. EEGs are often non-invasive with electrodes being placed on the skin of the subject. However, in some instances, electrodes are surgically implanted into the subject. For example, EEGs can be used to monitor a fetus in utero using a direct scalp measurement technique, which involves the insertion of fecal scalp electrodes through the birth canal and attaching it to the fetal scalp of the fetus.

### SUMMARY

In one embodiment, a computer-implemented method is provided. The method includes receiving a noninvasive transabdominal fetal electroencephalography (TA-fEEG) signal associated with a pregnant subject. The method further includes reducing unwanted noise in the TA-fEEG signal using a first machine learning model. The method further includes reconstructing a fetal electroencephalography (fEEG) signal from the TA-fEEG signal using a second machine learning model.

In addition to one or more of the features described herein, or as an alternative, further embodiments of the method may include that the noninvasive TA-fEEG signal is collected from a sensor of a noninvasive sensing device associated with the pregnant subject.

In addition to one or more of the features described herein, or as an alternative, further embodiments of the method may include predicting, based at least in part on the reconstructed fEEG signal, a likelihood of fetal hypoxia of a fetus of the pregnant subject.

In addition to one or more of the features described herein, or as an alternative, further embodiments of the method may include that the first machine learning model is a first neural network, and wherein the second machine learning model is a second neural network.

In addition to one or more of the features described herein, or as an alternative, further embodiments of the method may include training the first machine learning model.

In addition to one or more of the features described herein, or as an alternative, further embodiments of the method may include that the first machine learning model is trained using real scalp electroencephalography (EEG) data from premature babies, as ground truth data, in combination with a realistic volume conductor model of material and fetal tissue developed for fetal electrocardiography (ECG).

In addition to one or more of the features described herein, or as an alternative, further embodiments of the method may include training the second machine learning model.

In addition to one or more of the features described herein, or as an alternative, further embodiments of the method may include that the second machine learning model is trained using real scalp electroencephalography (EEG) data from premature babies, as ground truth data, in combination with a realistic volume conductor model of material and fetal tissue developed for fetal electrocardiography (ECG).

In addition to one or more of the features described herein, or as an alternative, further embodiments of the method may include that the first machine learning model is a deep neural network, and wherein the second machine learning model is an independent component analysis model.

In another embodiment a system for noninvasive transabdominal fetal electroencephalography (TA-fEEG) includes a noninvasive sensing device having a sensor to detect TA-fEEG signals of a pregnant subject and a processing system in communication with the sensor. The processing system includes a memory for storing computer readable instructions and a processing device for executing the computer readable instructions. The computer readable instructions controlling the processing device to perform operations. The operations include removing, using a first machine learning model, artefacts from the TA-fEEG signals, the artefacts caused by maternal and fetal cardiac activity and movement. The operations further include separating, using a second machine learning model, independent sources of activity embedded in the TA-fEEG signals. The operations further include predicting, based at least in part on at least one of the independent sources of activity embedded in the TA-fEEG signals, a likelihood of fetal hypoxia of a fetus of the pregnant subject.

In addition to one or more of the features described herein, or as an alternative, further embodiments of the system may include that the first machine learning model is a first neural network, and wherein the second machine learning model is a second neural network.

In addition to one or more of the features described herein, or as an alternative, further embodiments of the system may include, that the operations further include training the first machine learning model.

In addition to one or more of the features described herein, or as an alternative, further embodiments of the system may include that the first machine learning model is trained using real scalp electroencephalography (EEG) data from premature babies, as ground truth data, in combination with a realistic volume conductor model of material and fetal tissue developed for fetal electrocardiography (ECG).

In addition to one or more of the features described herein, or as an alternative, further embodiments of the system may include that the operations further include training the second machine learning model.

In addition to one or more of the features described herein, or as an alternative, further embodiments of the system may include that the second machine learning model is trained using real scalp electroencephalography (EEG) data from premature babies, as ground truth data, in combination with a realistic volume conductor model of material and fetal tissue developed for fetal electrocardiography (ECG).

In addition to one or more of the features described herein, or as an alternative, further embodiments of the system may include that the first machine learning model is a deep neural network, and wherein the second machine learning model is an independent component analysis model.

In yet another embodiment, a computer-implemented method for training machine learning models is provided. The method includes training a first machine learning model to reduce unwanted noise in a noninvasive transabdominal fetal electroencephalography (TA-fEEG) signal. The method further includes training a second machine learning model to reconstruct a fetal electroencephalography (fEEG) signal from the TA-fEEG signal.

In addition to one or more of the features described herein, or as an alternative, further embodiments of the method may include that the first machine learning model is trained using real scalp electroencephalography (EEG) data from premature babies, as ground truth data, in combination with a realistic volume conductor model of material and fetal tissue developed for fetal electrocardiography (ECG).

In addition to one or more of the features described herein, or as an alternative, further embodiments of the method may include that the second machine learning model is trained using real scalp electroencephalography (EEG) data from premature babies, as ground truth data, in combination with a realistic volume conductor model of material and fetal tissue developed for fetal electrocardiography (ECG).

In addition to one or more of the features described herein, or as an alternative, further embodiments of the method may include that the first machine learning model is a deep neural network, and wherein the second machine learning model is an independent component analysis model.

The above features and advantages, and other features and advantages, of the disclosure are readily apparent from the following detailed description when taken in connection with the accompanying drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of one or more embodiments described herein are apparent from the following detailed description taken in conjunction with the accompanying drawings in which:

FIG. 1A depicts a block diagram of a system for noninvasive transabdominal fetal electroencephalography according to one or more embodiments described herein;

FIG. 1B depicts a noninvasive sensing device according to one or more embodiments described herein;

FIG. 1C depicts the noninvasive sensing device of FIG. 1B being worn by a subject according to one or more embodiments described herein;

FIG. 2 depicts a block diagram of components of a machine learning training and inference system according to one or more embodiments described herein;

FIG. 3 depicts a flow diagram of a method for noninvasive transabdominal fetal electroencephalography according to one or more embodiments described herein;

FIG. 4 depicts a comparison of a neonatal EEG with a reconstructed transabdominal fEEG generated using machine learning according to one or more embodiments described herein; and

FIG. 5 depicts a block diagram of a processing system for implementing one or more embodiments described herein.

The diagrams depicted herein are illustrative. There can be many variations to the diagram or the operations described therein without departing from the scope of the embodiments described herein. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

### DETAILED DESCRIPTION

One or more embodiments described herein provide for noninvasive transabdominal fetal electroencephalography (TA-fEEG).

Reduced fetal brain oxygenation, known as fetal brain hypoxia, in utero can have devastating consequences, including irreversible neural compromise and even death. Fetal assessment aims to identify whether a fetus is at risk of fetal brain hypoxia in order to implement timely interventions (e.g., emergency C-section) to prevent harm to the fetus. Conventional approaches to fetal assessment, such as external fetal monitoring (EFM) and biophysical profile (BPP), utilize ultrasound-based techniques to measure downstream bodily responses to fetal brain hypoxia. However, these approaches reflect changes that occur after the onset of fetal brain hypoxia, which may explain why the rates of conditions associated with fetal brain hypoxia (e.g., cerebral palsy and fetal mortality) have not decreased despite widespread use of EFM and BPP during the past four decades.

An alternative and more sensitive approach to detecting early signs of fetal brain hypoxia is to monitor the changes in the fetal brain itself as captured in the electrical activity of the fetal brain. In fact, early studies conducted during childbirth using an intravaginal electrode attached to the fetal scalp showed that hypoxia-induced changes in the electrical activity of the fetal brain can precede changes in heart rate by up to 10 minutes. Detecting the early signs of fetal brain hypoxia by observing fetal brain activity would provide for earlier interventions to be implemented to reduce the risk of fetal harm. Conventionally, the only techniques available for measuring the electrical activity of the fetal brain in utero are fetal magnetoencephalography (fMEG) and direct fetal scalp electrodes (FSEs) inserted intravaginally. Unfortunately, these techniques have significant limitations that preclude them from widespread adoption.

fMEG scanners are not widely available due to their high cost. Further, fMEG scanners are not suitable for monitoring a fetus during childbirth. For example, for fMEG scanners to operate, pregnant women sit on a saddle-like seat while placing their abdomen inside the scanner. This design blocks the clinicians' access to the maternal abdomen and birth canal, which is crucial during childbirth.

Direct scalp measurement is an invasive technique involving the insertion of an FSE through the birth canal and attaching it to the fetal scalp. This procedure carries risks for both the mother and fetus, such as abrasion or lesions to the mother's birth canal and uterus, as well as potential damage to the fetal scalp. Furthermore, the amniotic sac is ruptured in order to place an FSE, thus limiting its use to childbirth and making FSEs unsuitable for monitoring fetal status and development during gestation.

Accordingly, while conventional techniques for fetal monitoring are suitable for their intended purposes, there is a need for non-invasive, unobstructed, and cost-effective approaches to accurately measure fetal brain activity throughout the stages of gestation and childbirth in order to provide medical providers with direct insight into the well-being of the developing fetus.

The above-described aspects address the shortcomings of the prior art by providing for a noninvasive transabdominal fetal EEG (fEEG). One or more embodiments described herein use a noninvasive TA-fEEG to measure electrical activity of a fetal brain. Similar to scalp electroencephalography (EEG), which is an inexpensive and widely used technique in which electrodes are attached to the human scalp are used to detect voltage changes generated by the human brain, in TA-fEEG electrodes are attached to the maternal abdomen and are used to detect voltage changes generated by the fetal brain. TA-fEEG signals are masked by a significant amount of high-amplitude artifactual activity resulting from maternal abdominal muscles, maternal and fetal cardiac activity, fetal movements, sweat-related drifts, uterine activity, and/or the like including combinations and/or multiples thereof. One or more techniques described herein apply artificial intelligence (AI) and machine learning (ML) techniques to reduce or eliminate unwanted noise in TA-fEEG signals and then reconstructing fetal EEG (fEEG) signals from data collected on the maternal abdomen. That is, one or more embodiments described herein provide for denoising electrical signals collected with electrodes attached to the material abdomen and then reconstructing fEEG signals.

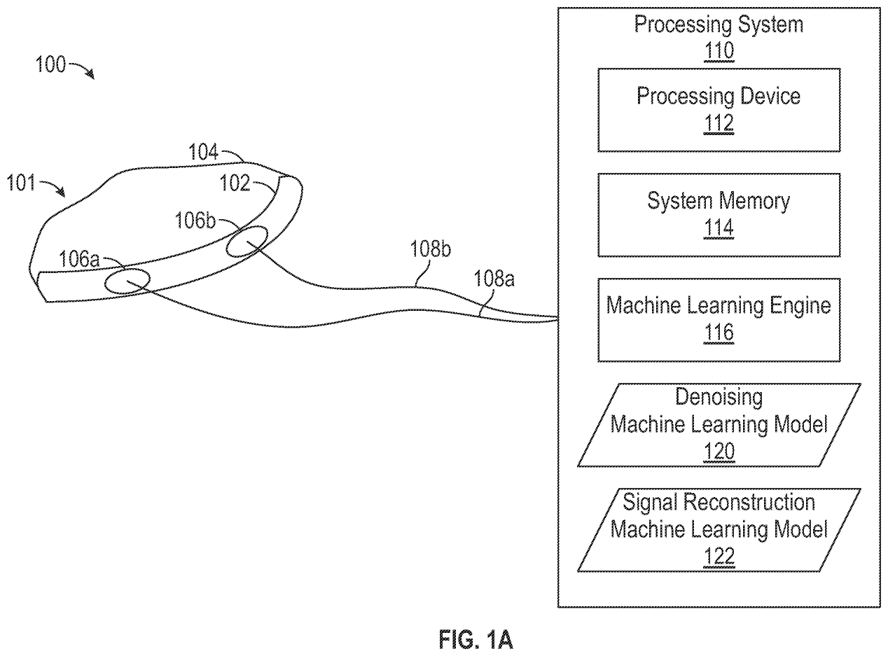

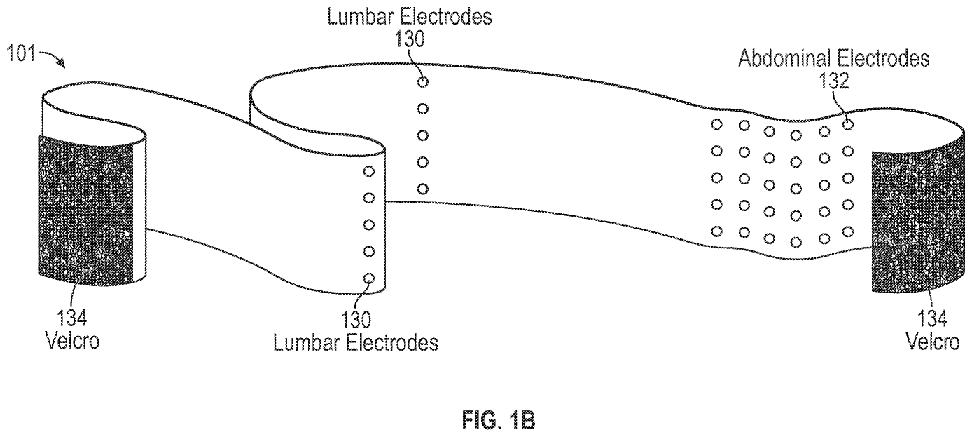

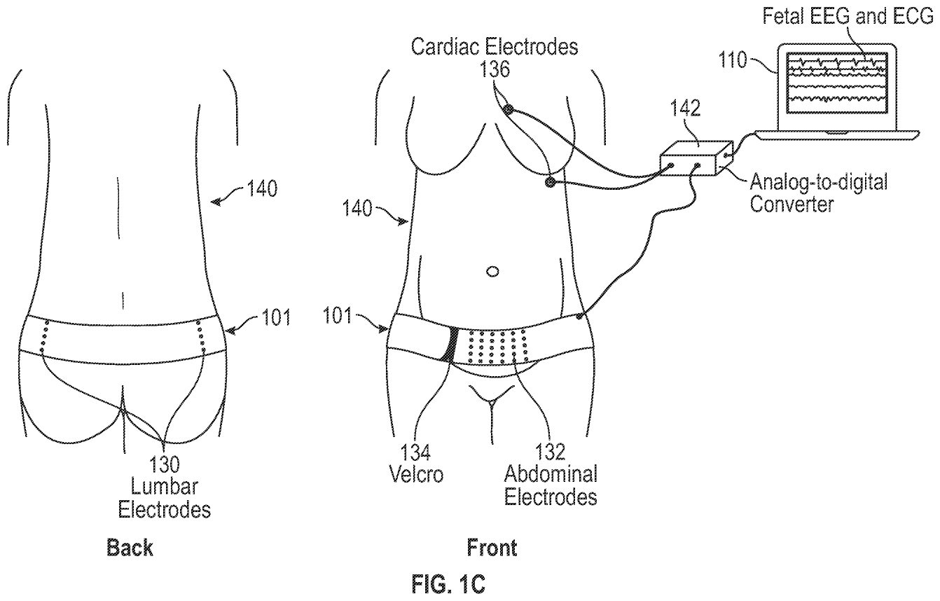

Turning now to FIG. 1A, a block diagram of a system 100 for noninvasive transabdominal fetal EEG is provided according to one or more embodiments described herein. The system 100 includes a sensing device 102 in communication with a processing system 110.

The sensing device 102 includes a noninvasive sensing device 101 that includes a sensor portion 102 and a connecting portion 104. The noninvasive sensing device 101 is configured to be worn by a pregnant woman, such as around the abdominal region. The sensor portion 102 of the noninvasive sensing device 101 includes one or more sensors 106a, 106b. Although two sensors 106a, 106b are shown, other numbers of sensors can be used in other embodiments. The connection portion 104 connects to the sensor portion 102 to enable a pregnant woman to wear the noninvasive sensing device 101 such that the noninvasive sensing device 101 can be positioned about the maternal abdomen. The noninvasive sensing device 101 can be adjustable to accommodate different sizes, orientations, configurations, and/or the like including combinations and/or multiples thereof. The one or more sensors 106a, 106b can be used to collect data about the pregnant woman and the fetus. The one or more sensors 106a, 106b can be electrodes configured to collect EEG data, for example.

The one or more sensors 106a, 106b of the noninvasive sensing device 101 can transmit data, such as EEG data, to the processing system 110 via one or more communication links 108a, 108b. Although two communication links 108a, 108b are shown, other numbers of sensors can be used in other embodiments. For example, the sensors 106a, 106b can share a communication link according to an embodiment. The communication links 108a, 108b can be wired and/or wireless links and may be configured to transmit analog signals and/or digital data.

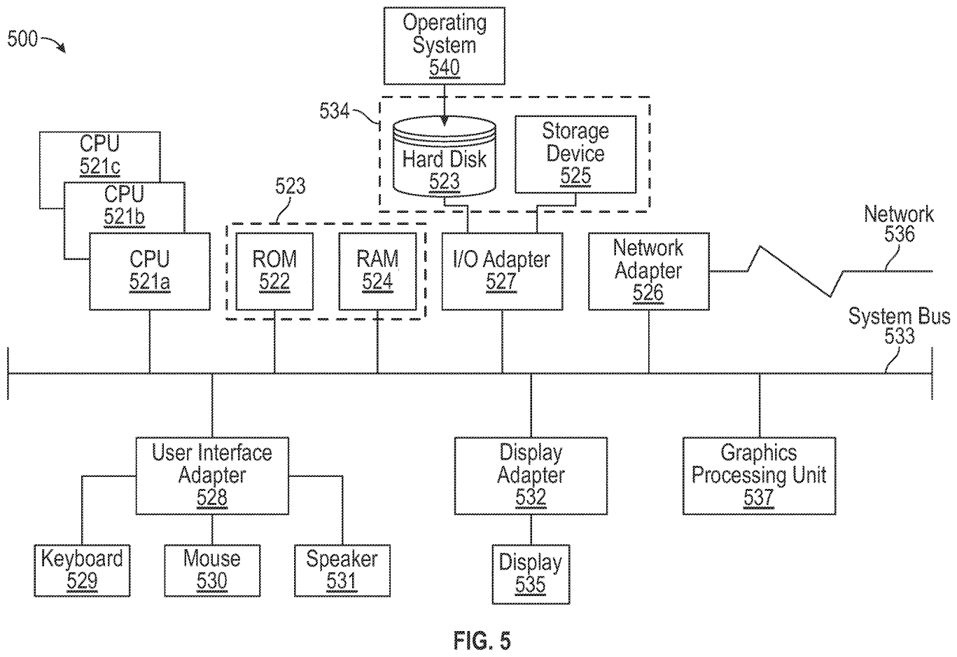

The processing system 110 includes a processing device 112, a system memory 114, and a machine learning (ML) engine 116. The various components, modules, engines, etc. (e.g., the machine learning engine 116) described regarding the processing system 110 can be implemented as instructions stored on a computer-readable storage medium, as hardware modules, as special-purpose hardware (e.g., application specific hardware, application specific integrated circuits (ASICs), application specific special processors (ASSPs), field programmable gate arrays (FPGAs), as embedded controllers, hardwired circuitry, etc.), or as some combination or combinations of these. According to aspects of the present disclosure, the engine(s) described herein can be a combination of hardware and programming. The programming can be processor executable instructions stored on a tangible memory, and the hardware can include the processing device 112 (e.g., the processing device(s) 521 of FIG. 5) for executing those instructions. Thus a system memory 114 (e.g., the system memory 523 of FIG. 5) can store program instructions that when executed by the processing device 112 implement the engines described herein. Other engines can also be utilized to include other features and functionality described in other examples herein.

The ML engine 116 can create and/or use one or more machine learning models, such as the denoising ML model 120, a signal reconstruction ML model 122, and/or the like including combinations and/or multiples thereof. Although the denoising ML model 120 and the signal reconstruction ML model 122 are shown as part of the processing system 110, it should be appreciated that one or more of these models can be stored on a remote processing system (e.g., another processing system, a node of a cloud computing system, and/or the like including combinations and/or multiples thereof) and accessed by a network connection. According to one or more embodiments described herein, a cloud computing system can be in wired or wireless electronic communication with the processing system 110. Cloud computing can supplement, support, or replace some or all of the functionality of the elements of the processing system 110. Some or all of the functionality of the elements of processing system 110 can be implemented as a node of a cloud computing system. For example, one or more of the denoising ML model 120 and the signal reconstruction ML model 122 can be stored on a node of a cloud computing system and accessed via the Internet. As another example, the machine learning engine 116 can be implemented using a cloud computing system, where the cloud computing system perform training and/or inference as described herein.

One or more embodiments described herein can utilize machine learning techniques to perform tasks, such as noninvasive transabdominal fetal electroencephalography. More specifically, one or more embodiments described herein can incorporate and utilize rule-based decision making and artificial intelligence (AI) reasoning to accomplish the various operations described herein, namely noninvasive TA-fEEG. The phrase “machine learning” broadly describes a function of electronic systems that learn from data. A machine learning system, engine, or module can include a trainable machine learning algorithm that can be trained, such as in an external cloud environment, to learn functional relationships between inputs and outputs, and the resulting model (sometimes referred to as a “trained neural network,” “trained model,” and/or “trained machine learning model”) can be used for noninvasive TA-fEEG, for example. In one or more embodiments, machine learning functionality can be implemented using an artificial neural network (ANN) having the capability to be trained to perform a function. In machine learning and cognitive science, ANNs are a family of statistical learning models inspired by the biological neural networks of animals, and in particular the brain. ANNs can be used to estimate or approximate systems and functions that depend on a large number of inputs. Convolutional neural networks (CNN) are a class of deep, feed-forward ANNs that are particularly useful at tasks such as, but not limited to analyzing visual imagery and natural language processing (NLP). Recurrent neural networks (RNN) are another class of deep, feed-forward ANNs and are particularly useful at tasks such as, but not limited to, unsegmented connected handwriting recognition and speech recognition. Other types of neural networks are also known and can be used in accordance with one or more embodiments described herein.

ANNs can be embodied as so-called “neuromorphic” systems of interconnected processor elements that act as simulated “neurons” and exchange “messages” between each other in the form of electronic signals. Similar to the so-called “plasticity” of synaptic neurotransmitter connections that carry messages between biological neurons, the connections in ANNs that carry electronic messages between simulated neurons are provided with numeric weights that correspond to the strength or weakness of a given connection. The weights can be adjusted and tuned based on experience, making ANNs adaptive to inputs and capable of learning. For example, an ANN for handwriting recognition is defined by a set of input neurons that can be activated by the pixels of an input image. After being weighted and transformed by a function determined by the network's designer, the activation of these input neurons are then passed to other downstream neurons, which are often referred to as “hidden” neurons. This process is repeated until an output neuron is activated. The activated output neuron determines which character was input. It should be appreciated that these same techniques can be applied in the case of noninvasive TA-fEEG as described herein.

The processing system 110 provides for extracting fEEG signals from signals collected noninvasively from the maternal abdomen and for reconstructing TA-fEEG by using machine learning techniques. According to one or more embodiments described herein, a first machine learning model (e.g., the denoising ML model 120), which can be a deep neural network, is used to remove artefacts resulting from maternal and fetal cardiac activity and movement, and a second machine learning model (e.g., the signal reconstruction ML model 122), which can be an independent component analysis (ICA) is used to separate independent sources of activity embedded in the signals. Signals resulting from the fetal brain can be used based on their morphology and spectrograms. This approach provides for measuring fetal neurologic activity as a more sensitive and earlier detection of fetal hypoxia in the pre-labor or labor period as compared to conventional approaches for detecting fetal hypoxia. Moreover, this approach is noninvasive.

FIGS. 1B and 1C depict another example of the noninvasive sensing device 101 according to one or more embodiments described herein. In this example, the noninvasive sensing device 101 includes lumbar electrodes 130 and abdominal electrodes 132. The noninvasive sensing device 101 can be connected around a subject 140, as shown in FIG. 1C. According to one or more embodiments described herein, the noninvasive sensing device 101 can be adjustably worn by the subject 140, such as using a hook-and-loop closure system (e.g., VELCRO® 134). As further shown in FIG. 1C, the lumbar electrodes 130 and/or the abdominal electrodes 132 can be connected to the processing system 110 via an analog-to-digital converter (ADC) 142. However, in other embodiments, the ADC 142 can be omitted. Cardiac electrodes 136 can also be connected to the processing system 110, such as via the ADC 142. The processing system can receive and/or process fEEG and electrocardiogramals received from the lumbar electrodes 130, abdominal electrodes 132, and/or the cardiac electrodes 136.

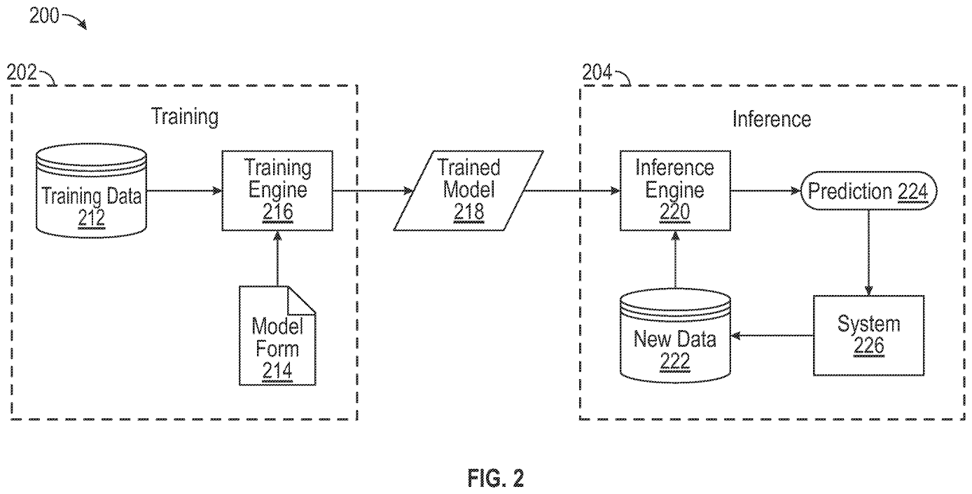

Systems for training and using a machine learning model are now described in more detail with reference to FIG. 2. Particularly, FIG. 2 depicts a block diagram of components of a machine learning training and inference system 200 according to one or more embodiments described herein. The system 200 performs training 202 and inference 204. During training 202, a training engine 216 trains a model (e.g., the trained model 218) to perform a task, such as to perform noninvasive TA-fEEG. It should be appreciated that the trained model 218 can represent one or more trained machine learning models, such as the denoising ML model 120 and/or the signal reconstruction ML model 122. In some cases, the training engine 216 trains multiple machine learning models, such as the denoising ML model 120 and the signal reconstruction ML model 122. According to one or more embodiments, the denoising ML model 120 can be trained to remove artefacts resulting from maternal and fetal cardiac activity and movement. According to one or more embodiments, the signal reconstruction ML model 122 can be trained to separate the independent sources of activity embedded in the TA-fEEG signals. According to one or more embodiments described herein, the denoising ML model 120 can be a first neural network and the signal reconstruction ML model 122 can be a second neural network. Inference 204 is the process of implementing the trained model 218 to perform the task, such as to perform noninvasive TA-fEEG, in the context of a larger system (e.g., a system 226). All or a portion of the system 200 shown in FIG. 2 can be implemented, for example by all or a subset of the processing system 110 of FIG. 1A.

The training 202 begins with training data 212, which may be structured or unstructured data. According to one or more embodiments described herein, the training data 212 for training the denoising ML model 120 and/or the for training the signal reconstruction ML model 122 includes real scalp EEG data from premature babies, which can be used as ground truth data, in combination with a realistic volume conductor model of material and fetal tissue (e.g., fat, muscle, amniotic sac, vernix caseosa, fetal head and brain, and/or the like including combinations and/or multiples thereof) developed for fetal ECG. The realistic volume conductor model of material and fetal tissue in combination with the EEG data from premature babies simulates fEEG signals on the maternal abdomen. During the training 202, the fEEG signals can be augmented by randomly scaling their amplitude and added to augmented artifactual signals (e.g., distorted and scaled low frequency noise, muscle activity, fetal and maternal ECG, fetal movement, and/or the like including combinations and/or multiples thereof).

The training engine 216 receives the training data 212 and a model form 214. The model form 214 represents a base model that is untrained. The model form 214 can have preset weights and biases, which can be adjusted during training. It should be appreciated that the model form 214 can be selected from many different model forms depending on the task to be performed. For example, where the training 202 is to train a model to perform image classification, the model form 214 may be a model form of a CNN. The training 202 can be supervised learning, semi-supervised learning, unsupervised learning, reinforcement learning, and/or the like, including combinations and/or multiples thereof. For example, supervised learning can be used to train a machine learning model to classify an object of interest in an image. To do this, the training data 212 includes labeled images, including images of the object of interest with associated labels (ground truth) and other images that do not include the object of interest with associated labels. In this example, the training engine 216 takes as input a training image from the training data 212, makes a prediction for classifying the image, and compares the prediction to the known label. The training engine 216 then adjusts weights and/or biases of the model based on results of the comparison, such as by using backpropagation. The training 202 may be performed multiple times (referred to as “epochs”) until a suitable model is trained (e.g., the denoising ML model 120 and/or the signal reconstruction ML model 122).

The training 202 can include both training and validating the trained model 218 (e.g., one or more of the denoising ML model 120 and/or the signal reconstruction ML model 122). For example, the denoising ML model 120 and/or the signal reconstruction ML model 122 can be trained and validated using augmented data and tested on real data collected from pregnant subjects. According to one or more embodiments described herein, training and/or validating one or more of the denoising ML model 120 and/or the signal reconstruction ML model 122 can be performed using synthetic data generated based on real data collected from pregnant subjects.

Once trained, the trained model 218 can be used to perform inference 204 to perform a task, such as to reconstruct fEEG and remove “unwanted” signals. The inference engine 220 applies the trained model 218 to new data 222 (e.g., real-world, non-training data). For example, in the case of TA-fEEG, the new data 222 can be TA-fEEG data captured about a subject noninvasively as described herein, the new data 222 not having been part of the training data 212. In this way, the new data 222 represents data to which the model 218 has not been exposed. The inference engine 220 makes a prediction 224 (e.g., predicting, based at least in part on the reconstructed fEEG signal, a likelihood of fetal hypoxia of a fetus of the pregnant subject using the new data 222) and passes the prediction 224 to the system 226 (e.g., the processing system 110 of FIG. 1A). The system 226 can, based on the prediction 224, taken an action, perform an operation, perform an analysis, and/or the like, including combinations and/or multiples thereof. In some embodiments, the system 226 can add to and/or modify the new data 222 based on the prediction 224.

In accordance with one or more embodiments, the predictions 224 generated by the inference engine 220 are periodically monitored and verified to ensure that the inference engine 220 is operating as expected. Based on the verification, additional training 202 may occur using the trained model 218 as the starting point. The additional training 202 may include all or a subset of the original training data 212 and/or new training data 212. In accordance with one or more embodiments, the training 202 includes updating the trained model 218 to account for changes in expected input data.

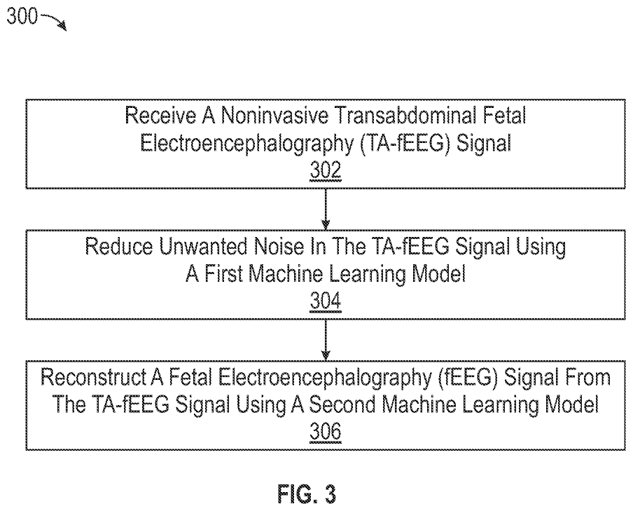

FIG. 3 depicts a flow diagram of a method 300 for noninvasive transabdominal fetal electroencephalography according to one or more embodiments described herein. The method 300 can be performed by any suitable system or device, such as the system 100 of FIG. 1A, the processing system 110 of FIG. 1A, the machine learning training and inference system 200 of FIG. 2, the processing system 500 of FIG. 5, and/or the like including combinations and/or multiples thereof. The method 300 is now described with reference to FIG. 1A but is not so limited.

At block 302, the processing system 110 receives, from the noninvasive sensing device 101, a noninvasive TA-fEEG signal associated with a pregnant subject. At block 304, the machine learning engine 116, using the denoising ML model 120, reduces (or eliminates) unwanted noise in the TA-fEEG signal. At block 306, the machine learning engine 116, using the signal reconstruction ML model 122, reconstructs an fEEG signal from the TA-fEEG signal.

According to one or more embodiments described herein, the noninvasive TA-fEEG signal is collected from a sensor of a noninvasive sensing device associated with the pregnant subject.

According to one or more embodiments described herein, the method 300 can include predicting, based at least in part on the reconstructed fEEG signal, a likelihood of fetal hypoxia of a fetus of the pregnant subject.

According to one or more embodiments described herein, the first machine learning model is a first neural network, and wherein the second machine learning model is a second neural network.

According to one or more embodiments described herein, the method 300 can include training the denoising ML model 120 as described herein. For example, the denoising ML model 120 can be trained using real scalp EEG data from premature babies, as ground truth data, in combination with a realistic volume conductor model of material and fetal tissue developed for ECG.

According to one or more embodiments described herein, the method 300 can include training the signal reconstruction ML model 122 as described herein. For example, the signal reconstruction ML model 122 can be trained using real scalp EEG data from premature babies, as ground truth data, in combination with a realistic volume conductor model of material and fetal tissue developed for ECG.

According to one or more embodiments described herein, the denoising ML model 120 is a deep neural network, and the signal reconstruction ML model 122 is an independent component analysis model.

Additional processes also may be included, and it should be understood that the process depicted in FIG. 3 represents an illustration, and that other processes may be added or existing processes may be removed, modified, or rearranged without departing from the scope of the present disclosure.

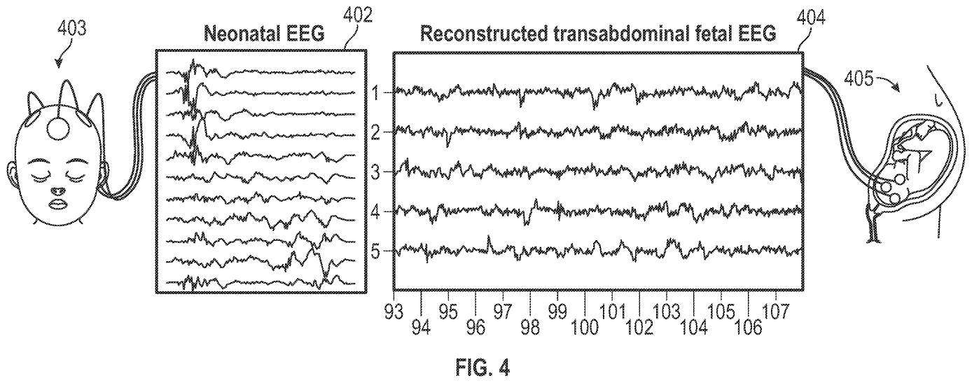

FIG. 4 depicts a comparison of a neonatal EEG 402 with a reconstructed transabdominal fetal EEG 404 generated using machine learning according to one or more embodiments described herein. The neonatal EEG 402 is generated from electrodes attached to, for example, the scalp of a premature baby 403. The TA-fEEG 404 is generated from electrodes attached to a fetus in utero 405. In this example, the premature baby 403 and the fetus in utero 405 are aged matched, meaning that the premature baby 403 and the fetus in utero 405 are of substantially the same gestational age. The signal from the electrodes attached to the fetus in utero 405 is processed using the denoising ML model 120 and the signal reconstruction ML model 122 according to one or more embodiments described herein in order to generate the reconstructed TA-fEEG 404. The denoising ML model 120 and the signal reconstruction ML model 122 were able to reconstruct fEEG data and remove unwanted signals. The non-invasive TA-fEEG 404 can be compared to the age-matched neonatal direct scalp electrode EEG (e.g., the neonatal EEG 402). That is, the reconstructed TA-fEEG 404 can be compared to the neonatal EEG 402, which can be generated, for example, using aged-matched fetal magnetoencephalography (fMEG) data. Both the reconstructed TA-fEEG 404 and the neonatal EEG 402 show characteristic signal patters of the gestational age with discontinuous patters of neurologic activity. Auditory stimuli with event-related potential (e.g., specific deflections in the brain's electrical potential that occurs in response to stimuli) can be observed from the non-invasive collection and compared to equivalent measures in fMEG.

According to one or more embodiments described herein, a possible use case for TA-fEEG is for fetal hypoxia prediction. Fetal hypoxia is difficult to detect. Electronic fetal monitoring relies on downstream effects of hypoxia on fetal cardiac activity, limiting its accuracy and timeliness. For example, a conventional approach for fetal hypoxia prediction is as follows: oxygenated blood enters the placenta, oxygen exchange occurs at the intervillous space, fetal neurologic system (parasympathetic and sympathetic system) reacts to oxygen status, and electronic fetal monitoring shows heart rate changes. This process is slow and error prone. One or more embodiments described herein improve the timeliness and accuracy of conventional approaches to fetal hypoxia detection by applying trained machine learning models to TA-fEEG signals to target the upstream effects of neurological activity earlier for more accurate detection of fetal hypoxia. Use of the denoising ML model 120 and the signal reconstruction ML model 122 provide for reliably measuring fetal EEG signals, leading to earlier detection of fetal hypoxia compared to conventional approaches (e.g., using downstream approaches relating to cardiac activity). The TA-fEEG approach described herein provides many possible clinical implications.

For an in-labor scenario, there is a high chance of fetal hypoxia and/or injury. One or more embodiments described herein provide for an earlier detector of fetal hypoxia, increased sensitive monitoring, earlier intervention, decreased rates of cerebral palsy in labor, decreased rates of unnecessary cesarean section, improved outcomes for both mother and baby as a result of revolutionized labor monitoring, and/or the like including combinations and/or multiples thereof. For a pre-labor scenario, high-risk fetuses can be targeted to provide additional clinical information. One or more embodiments described herein provide for targeting fetuses with congenital anomalies such as brain anomalies or spina bifida, provides neurologic outlook and expectations at birth for parents, provide auditory stimuli as a first hearing test for at-risk fetuses for congenital hearing loss, and/or the like including combinations and/or multiples thereof. It should be appreciated that other in-labor and pre-labor use cases for the one or more embodiments described herein are also possible and are not limited by the examples provided.

Regarding cerebral palsy (CP), CP is a common motor disorder in childhood with lifelong symptoms of mobility impairment, developmental delay, chronic pain, seizure disorder, and/or the like including combinations and/or multiples thereof. Children with cerebral palsy incur approximately 26 times the cost of medical care, and national lifetime costs for all children born with cerebral palsy in 2000 is estimated to be $11.5 billion. Many CP cases are congenital and occur in the prenatal or birth period due to unrecognized fetal compromise. Electronic fetal monitoring via heart rate patterns can be used in over 90% of labors but despite its widespread use, the rate of cerebral palsy has not decreased and instead, the rate of cesarean section and its associated maternal risks and costs to healthcare has steadily increased since the adoption of this technology. This illustrates the current costs (e.g., financial, health, long-term risk) of an inadequate fetal monitoring system and the need for more sensitive and accurate monitoring for fetal hypoxia. Fetal EEG has been shown to be more accurate and more timely, allowing for earlier and accurate intervention for fetal compromise, with the goal of decreasing the rates of CP and decreasing the rates of unnecessary cesarean delivery.

It is understood that one or more embodiments described herein is capable of being implemented in conjunction with any other type of computing environment now known or later developed. For example, FIG. 5 depicts a block diagram of a processing system 500 for implementing the techniques described herein. In accordance with one or more embodiments described herein, the processing system 500 is an example of a cloud computing node of a cloud computing system. In examples, processing system 500 has one or more central processing units (“processors” or “processing resources” or “processing devices”) 521a, 521b, 521c, etc. (collectively or generically referred to as processor(s) 521 and/or as processing device(s) 521). In aspects of the present disclosure, each processor 521 can include a reduced instruction set computer (RISC) microprocessor. Processors 521 are coupled to system memory (e.g., random access memory (RAM) 524) and various other components via a system bus 533. Read only memory (ROM) 522 is coupled to system bus 533 and may include a basic input/output system (BIOS), which controls certain basic functions of processing system 500. A system memory 523 can include the ROM 522, the RAM 524, and/or any other suitable memory device including combinations and/or multiples thereof.

Further depicted arc an input/output (I/O) adapter 527 and a network adapter 526 coupled to system bus 533. I/O adapter 527 may be a small computer system interface (SCSI) adapter that communicates with a hard disk 523 and/or a storage device 525 or any other similar component. I/O adapter 527, hard disk 523, and storage device 525 are collectively referred to herein as mass storage 534. Operating system 540 for execution on processing system 500 may be stored in mass storage 534. The network adapter 526 interconnects system bus 533 with an outside network 536 enabling processing system 500 to communicate with other such systems.

A display 535 (e.g., a display monitor) is connected to system bus 533 by display adapter 532, which may include a graphics adapter to improve the performance of graphics intensive applications and a video controller. In one aspect of the present disclosure, adapters 526, 527, and/or 532 may be connected to one or more I/O busses that are connected to system bus 533 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 533 via user interface adapter 528 and display adapter 532. A keyboard 529, mouse 530, and speaker 531 may be interconnected to system bus 533 via user interface adapter 528, which may include, for example, a Super I/O chip integrating multiple device adapters into a single integrated circuit.

In some aspects of the present disclosure, processing system 500 includes a graphics processing unit 537. Graphics processing unit 537 is a specialized electronic circuit designed to manipulate and alter memory to accelerate the creation of images in a frame buffer intended for output to a display. In general, graphics processing unit 537 is very efficient at manipulating computer graphics and image processing, and has a highly parallel structure that makes it more effective than general-purpose CPUs for algorithms where processing of large blocks of data is done in parallel.

Thus, as configured herein, processing system 500 includes processing capability in the form of processors 521, storage capability including system memory (e.g., RAM 524), and mass storage 534, input means such as keyboard 529 and mouse 530, and output capability including speaker 531 and display 535. In some aspects of the present disclosure, a portion of system memory (e.g., RAM 524) and mass storage 534 collectively store the operating system 540 to coordinate the functions of the various components shown in processing system 500.

Various embodiments are described herein with reference to the related drawings. Alternative embodiments can be devised without departing from the scope of the claims. Various connections and positional relationships (e.g., over, below, adjacent, etc.) are set forth between elements in the following description and in the drawings. These connections and/or positional relationships, unless specified otherwise, can be direct or indirect, and the embodiments described herein are not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

The flowchart and block diagrams in the figures illustrate the architecture, functionality, and operation of possible implementations of systems, methods, and computer program products according to various embodiments described herein. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method comprising:
receiving a noninvasive transabdominal fetal electroencephalography (TA-fEEG) signal associated with a pregnant subject;
reducing unwanted noise in the TA-fEEG signal using a first machine learning model; and
reconstructing a fetal electroencephalography (fEEG) signal from the TA-fEEG signal using a second machine learning model.

2. The computer-implemented method of claim 1, wherein the noninvasive TA-fEEG signal is collected from a sensor of a noninvasive sensing device associated with the pregnant subject.

3. The computer-implemented method of claim 1, further comprising predicting, based at least in part on the reconstructed fEEG signal, a likelihood of fetal hypoxia of a fetus of the pregnant subject.

4. The computer-implemented method of claim 1, wherein the first machine learning model is a first neural network, and wherein the second machine learning model is a second neural network.

5. The computer-implemented method of claim 1, further comprising training the first machine learning model.

6. The computer-implemented method of claim 5, wherein the first machine learning model is trained using real scalp electroencephalography (EEG) data from premature babies, as ground truth data, in combination with a realistic volume conductor model of material and fetal tissue developed for fetal electrocardiography (ECG).

7. The computer-implemented method of claim 1, further comprising training the second machine learning model.

8. The computer-implemented method of claim 7, wherein the second machine learning model is trained using real scalp electroencephalography (EEG) data from premature babies, as ground truth data, in combination with a realistic volume conductor model of material and fetal tissue developed for fetal electrocardiography (ECG).

9. The computer-implemented method of claim 1, wherein the first machine learning model is a deep neural network, and wherein the second machine learning model is an independent component analysis model.

10. A system for noninvasive transabdominal fetal electroencephalography (TA-fEEG), the system comprising:
a noninvasive sensing device comprising a sensor to detect TA-fEEG signals of a pregnant subject; and
a processing system in communication with the sensor, the processing system comprising a memory for storing computer readable instructions and a processing device for executing the computer readable instructions, the computer readable instructions controlling the processing device to perform operations comprising:
removing, using a first machine learning model, artefacts from the TA-fEEG signals, the artefacts caused by maternal and fetal cardiac activity and movement;
separating, using a second machine learning model, independent sources of activity embedded in the TA-fEEG signals; and
predicting, based at least in part on at least one of the independent sources of activity embedded in the TA-fEEG signals, a likelihood of fetal hypoxia of a fetus of the pregnant subject.

11. The system of claim 10, wherein the first machine learning model is a first neural network, and wherein the second machine learning model is a second neural network.

12. The system of claim 10, the operations further comprising training the first machine learning model.

13. The system of claim 12, wherein the first machine learning model is trained using real scalp electroencephalography (EEG) data from premature babies, as ground truth data, in combination with a realistic volume conductor model of material and fetal tissue developed for fetal electrocardiography (ECG).

14. The system of claim 10, the operations further comprising training the second machine learning model.

15. The system of claim 14, wherein the second machine learning model is trained using real scalp electroencephalography (EEG) data from premature babies, as ground truth data, in combination with a realistic volume conductor model of material and fetal tissue developed for fetal electrocardiography (ECG).

16. The system of claim 10, wherein the first machine learning model is a deep neural network, and wherein the second machine learning model is an independent component analysis model.

17. A computer-implemented method for training machine learning models, the method comprising:
training a first machine learning model to reduce unwanted noise in a noninvasive transabdominal fetal electroencephalography (TA-fEEG) signal; and
training a second machine learning model to reconstruct a fetal electroencephalography (fEEG) signal from the TA-fEEG signal.

18. The computer-implemented method of claim 17, wherein the first machine learning model is trained using real scalp electroencephalography (EEG) data from premature babies, as ground truth data, in combination with a realistic volume conductor model of material and fetal tissue developed for fetal electrocardiography (ECG).

19. The computer-implemented method of claim 17, wherein the second machine learning model is trained using real scalp electroencephalography (EEG) data from premature babies, as ground truth data, in combination with a realistic volume conductor model of material and fetal tissue developed for fetal electrocardiography (ECG).

20. The computer-implemented method of claim 17, wherein the first machine learning model is a deep neural network, and wherein the second machine learning model is an independent component analysis model.
